(19) United States
(12) Patent Application Publication
(10) Pub. No.: US 2023/0109797 A1
(43) Pub. Date: Apr. 13, 2023

(54) API HEALTH ANALYSIS

(71) Applicant: International Business Machines Corporation, Armonk, NY (US)

(72) Inventors: Manish Anand Bhide, Hyderabad (IN); Remus Lazar, Morgan Hill, CA (US); Ravi Chandra Chamarthy, Hyderabad (IN)

(21) Appl. No.: 17/479,393

(22) Filed: Sep. 20, 2021

(51) Int. Cl. G06F 9/54; G06F 11/30

(52) U.S. Cl. CPC G06F 9/547; G06F 11/302; G06F 11/3072

(57) ABSTRACT

Disclosed embodiments provide techniques for monitoring application program interface (API) health based on design-time checks and runtime performance. The health of an API can include multiple factors. The factors may include correctness of the data returned/output by the API. A high amount of incorrect data may correlate to a lower health metric. A low amount of incorrect data may correlate to a higher health metric. If a health metric value falls below a predetermined threshold, it may indicate that the API health is poor, and hence, should not be used, or should be used with caution, depending on the application. In embodiments, an alternate API may be automatically selected as a replacement for an API in response to a health metric falling below a predetermined threshold.

### FIELD

Disclosed embodiments relate generally to computer systems, and more particularly, to application programming interface (API) health analysis.

### BACKGROUND

Application programming interface API functions are a common technique for software developers to interface with services, without needing to be concerned with the details of the inner workings of that service or the service provider.

Application developers can use APIs to access services through web-based communication. A developer writes code that calls the provider’s API, passes the input parameters/arguments in the context of the call, and then examines the response to confirm the operation.

A cloud API enables end users to access a cloud provider’s application or service, such as compute infrastructure, storage resources or monitoring tools. APIs define the possible features and functions of that application or service, along with the details needed to execute them. APIs can be based on REST or Simple Object Access Protocol (SOAP), or other suitable protocol.

APIs are well-suited for applications that are implemented as multiple distributed components. The application components call upon each other’s API(s). In this way, the components can be updated, scaled independently and located in distributed locations for greater performance and availability. One such example of this is container-based microservices applications that rely on APIs for container-to-container communication.

Developers can quickly create powerful applications using APIs. For service providers, APIs allow them to maintain their control and security of their data. Furthermore, providers can also track API usage, which makes it a valuable and growing source of revenue. Thus, APIs play a critical role in the development and deployment of computer applications.

### SUMMARY

In one embodiment, there is provided a computer-implemented method, comprising: computing an application programming interface (API) health metric for an application programming interface function; reporting the API health metric to a remote computing device; determining if the API health metric is below a predetermined threshold; and generating a message in response to determining that the API health metric is below the predetermined threshold.

In another embodiment, there is provided an electronic computation device comprising: a processor; a memory coupled to the processor, the memory containing instructions, that when executed by the processor, cause the electronic computation device to: compute an application programming interface (API) health metric for an application programming interface function; report the API health metric to a remote computing device; determine if the API health metric is below a predetermined threshold; and generate a message in response to determining that the API health metric is below the predetermined threshold.

In yet another embodiment, there is provided a computer program product for an electronic computation device comprising a computer readable storage medium having program instructions embodied therewith, the program instructions executable by a processor to cause the electronic computation device to: compute an application programming interface (API) health metric for an application programming interface function; report the API health metric to a remote computing device; determine if the API health metric is below a predetermined threshold; and generate a message in response to determining that the API health metric is below the predetermined threshold.

### BRIEF DESCRIPTION OF THE DRAWINGS

In the drawings, like numbering may represent like elements. Furthermore, certain elements in some of the Figures may be omitted, or illustrated not-to-scale, for illustrative clarity.

FIG. 1 shows an environment for embodiments of the present invention.

FIG. 2 is a flowchart indicating process steps for embodiments of the present invention.

FIG. 3 shows an exemplary API health record in accordance with embodiments of the present invention.

FIG. 4 shows additional data structures in accordance with embodiments of the present invention.

FIG. 5 is a flowchart showing additional process steps for embodiments of the present invention.

FIG. 6 is an API dashboard user interface in accordance with embodiments of the present invention.

FIG. 7 is an API configuration user interface in accordance with embodiments of the present invention.

FIG. 8 is a block diagram of a client device in accordance with embodiments of the present invention.

The drawings are not necessarily to scale. The drawings are merely representations, not necessarily intended to portray specific parameters of the invention. The drawings are intended to depict only example embodiments of the invention, and therefore should not be considered as limiting in scope.

### DETAILED DESCRIPTION

Disclosed embodiments provide techniques for monitoring API health based on design-time checks and runtime performance. The health of an API can include multiple factors. The factors may include correctness of the data returned/output by the API. A high amount of incorrect data may correlate to a lower health metric (score). A low amount of incorrect data may correlate to a higher health metric. If a health metric value falls below a predetermined threshold, it may indicate that the API health is poor, and hence, should not be used, or should be used with caution, depending on the application. In some embodiments, an alternate API may be automatically selected as a replacement for an API in response to a health metric falling below a predetermined threshold.

The factors may include completeness of the data returned/output by the API. A high amount (or frequent occurrences) of incomplete data may correlate to a lower health metric (score). A low amount (or infrequent occurrences) of incomplete data may correlate to a higher health metric. The factors may include timeliness of the data returned/output by the API. A long response time for providing the output data may correlate to a lower health metric (score). A short response time for providing the output data may correlate to a higher health metric (score). The factors may include a return time of the API. A long return time of the API may correlate to a lower health metric (score). A short return time for the API may correlate to a higher health metric (score). In some embodiments, the health metric may be based on a numerical scale from zero to 100, with a higher value indicating a higher API health. Other numerical scales are possible in disclosed embodiments.

Reference throughout this specification to “one embodiment,” “an embodiment,” “some embodiments”, or similar language means that a particular feature, structure, or characteristic described in connection with the embodiment is included in at least one embodiment of the present invention. Thus, appearances of the phrases “in one embodiment,” “in an embodiment,” “in some embodiments”, and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope and purpose of the invention. Thus, it is intended that the present invention cover the modifications and variations of this invention provided they come within the scope of the appended claims and their equivalents. Reference will now be made in detail to the preferred embodiments of the invention.

The terminology used herein is for the purpose of describing particular embodiments only and is not intended to be limiting of this disclosure. As used herein, the singular forms “a”, “an”, and “the” are intended to include the plural forms as well, unless the context clearly indicates otherwise. Furthermore, the use of the terms “a”, “an”, etc., do not denote a limitation of quantity, but rather denote the presence of at least one of the referenced items. The term “set” is intended to mean a quantity of at least one. It will be further understood that the terms “comprises” and/or “comprising”, or “includes” and/or “including”, or “has” and/or “having”, when used in this specification, specify the presence of stated features, regions, integers, steps, operations, elements, and/or components, but do not preclude the presence or addition of one or more other features, regions, or elements.

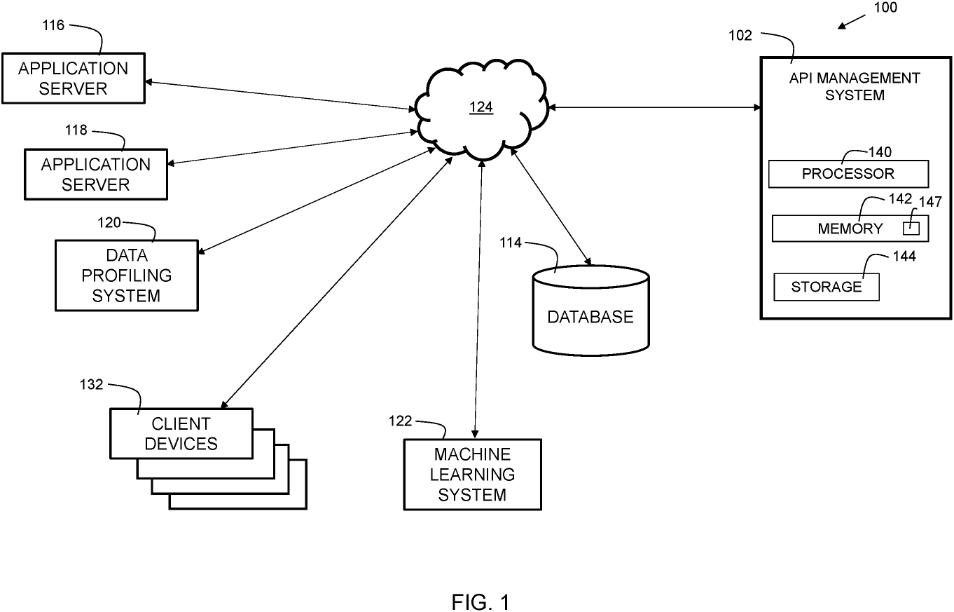

FIG. 1 is an environment 100 for embodiments of the present invention. At 102, there is an API management system (AMS). System 102 is an electronic computation device. System 102 includes a processor 140, memory 142, and storage 144. Memory 142 comprises instructions 147, which when executed by the processor, cause system 102 to implement embodiments of the invention. System 102 is in communication with network 124. Network 124 may be the internet, a wide area network, a local area network, a cloud network, or other suitable network.

One or more application servers may be connected to network 124. As shown in FIG. 1, there are two application servers, indicated as 116 and 118. Application servers 116 and 118 may each represent a computer, computing cluster, collection of virtual machines, containerized applications, or other collection of computers which may implement an API.

One or more client devices 132 may be connected to network 124. The client devices 132 may run applications that use APIs implemented on one or more application servers. The client devices 132 may invoke the APIs using HTTP (Hypertext Transfer Protocol), XML, JSON-RPC, REST, SOAP, or other suitable protocol.

Database 114 is connected to network 124. Database 114 may store information used by APIs. Database 114 may be a relational database, such as an SQL (Structured Query Language) database. Other database schema are possible in embodiments of the present invention.

Data profiling system 120 is connected to network 124. The data profiling system can perform integrity checks, purging of inaccurate, incomplete, duplicated, outdated, or unnecessary information. In embodiments, the data profiling system may be the IBM InfoSphere Information Analyzer, or other suitable data profiling system.

Also connected to network 124 is machine learning system 122. In some embodiments, machine learning system 122 is used to perform various functions in accordance with embodiments of the present invention. These functions can include, but are not limited to, estimating an API health metric based on trends, usage patterns, and/or other factors. The machine learning system 122 may utilize neural networks, convolutional neural networks (CNNs), and/or other suitable techniques.

In some embodiments, the AMS 102 is a standalone electronic computation device. It monitors deployed APIs to check for performance, data completeness, data accuracy, and/or other factors.

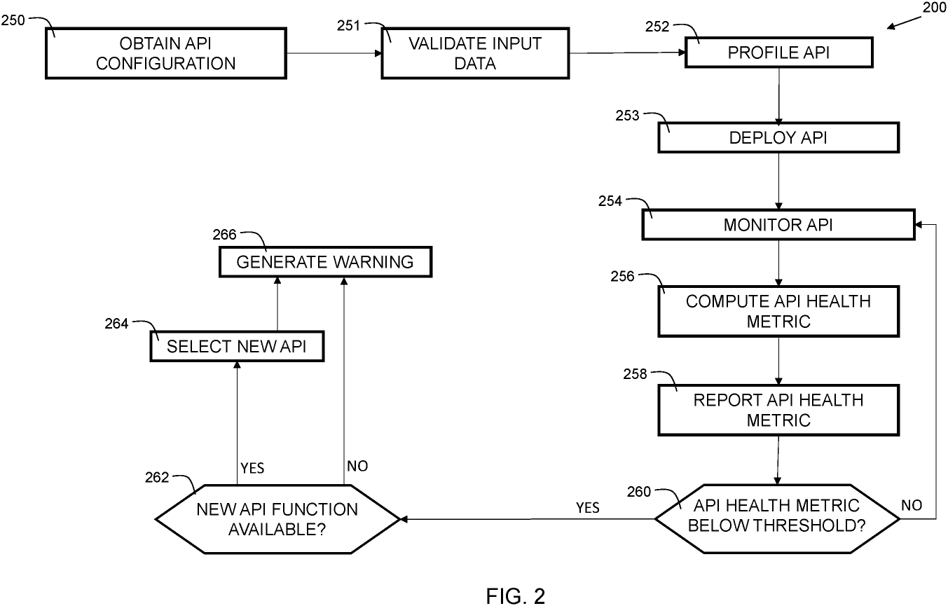

FIG. 2 is a flowchart 200 indicating process steps for embodiments of the present invention. At 250, an API configuration is obtained. The API configuration may be established by an application designer, and contains information such as health metric thresholds, and factors to be used in determining API health. At 251, input data is validated. This can include spot checking, or complete checking, of one or more columns of data within database 114. The data may be checked for completeness, accuracy, relevance, and/or other factors. As an example, a database column containing ZIP codes may be expected to have entries with 5-digit numbers. With this example, entries that are less than five digits, contain letters, or non-alphanumeric characters are considered to be invalid entries. The health metric is computed to be inversely related to the number of invalid entries. In this way, input data that is of low-quality results in a lower health metric for any APIs that use that input data. In embodiments, the input data validation may be performed utilizing a data profiling system such as that indicated at 120 in FIG. 1. Thus, embodiments can include performing an input data validation process.

At 252, the API is profiled. In embodiments, the API profiling may be performed by the AMS 102 of FIG. 1. The API profiling may comprise invoking the API with a variety of input data, and confirming the correctness, completeness, and/or timeliness of the resulting output data from the API. In some embodiments, an API input integrity metric may be computed. To compute an API input integrity metric, input errors may be intentionally used in the API arguments. If the API does not return an error in those situations, that may result in a lower API input integrity metric, and hence, a lower overall API health metric.

At 253, the API is deployed. When an API is deployed, it is made available for use by applications. In some embodiments, there can be various stages of deployment, such as beta testing, large scale testing, and/or general deployment.

At 254, the API is monitored. In some embodiments, all invocations of an API are monitored. In some embodiments, a subset of API invocations are monitored. The monitoring can include checking various factors such as correctness of data, completeness of data, timeliness of data, API return time, and an API success ratio. The API success ratio is a ratio of successful return statuses to the total number of invocations of the API.

At 256, an API health metric is computed. In embodiments, the API health metric J, indicative of overall API health, may be computed as follows:

$$\begin{matrix}{\text{J=K}}_{\text{1}} {\text{F}}_{\text{1}} \left(\text{D}\right) {\text{+K}}_{\text{2}} {\text{F}}_{\text{2}} \left(\text{E}\right) {\text{+K}}_{\text{3}} {\text{F}}_{\text{3}} \left(\text{O}\right) {\text{+K}}_{\text{4}} {\text{F}}_{\text{4}} \left(\text{C}\right) \text{+} \\ {\text{K}}_{\text{5}} {\text{F}}_{\text{5}} \left(\text{H}\right) {\text{+K}}_{\text{6}} {\text{F}}_{\text{6}} \left(\text{T}\right) {\text{+K}}_{\text{7}} {\text{F}}_{\text{7}} \left(\text{R}\right)\end{matrix}$$

Where:
Fi represents a mathematical function for the ith parameter;
Ki represents a constant for the ith parameter; and
Where the parameters are listed as follows:
   D is a measure of database health for the database 114, as determined by data profiling system 120;
   E is a measure of API input integrity, which includes a numerical value indicative of how well or poorly an API validates input data;
   O is a measure of API output correctness, which includes a numerical value indicative of how often or rarely an API produces incorrect output data;
   C is a measure of API output completeness, which includes a numerical value indicative of how often or rarely an API provides missing output data;
   H is a measure of API output data timeliness, which includes a numerical value indicative of how often or rarely an API produces timely output data; and
   R is an API success ratio, which includes a numerical value indicative of how often or rarely an API produces returns success upon being invoked.

At 258, the API health metric J, and/or the aforementioned parameters D, E, O, C, H, T, and/or R, are sent to a remote computing device. The remote computing device can include an application server such as 116 and 118 of FIG. 1, and/or client devices, such as indicated at 132 of FIG. 1. In some embodiments, one or more of the aforementioned parameters may be omitted in certain use cases. In some embodiments, the API health metric J may be normalized to a fixed scale, such as 0 -100, regardless of which factors are included/omitted in the computation of the API health metric J.

At 260, a check is made to determine if the API health metric J is below a predetermined level. In some embodiments, the API health metric J can range from 0 to 100, with 100 being a highest level of health and zero being a lowest level of health. In some embodiments, the predetermined threshold is selected in a range from 70 to 90. Other values are possible for the predetermined threshold in disclosed embodiments. If no at 260, then the process returns to 254 to periodically monitor the API. The monitoring period can range from seconds to minutes or hours, based on the application. In some embodiments, the monitoring period may be in the range of 30 seconds to 120 seconds. In some embodiments, the monitoring period may be in the range of ten minutes to 30 minutes. In some embodiments, the monitoring period may be in the range of two hours to six hours. In some embodiments, the monitoring period may be another suitable duration.

If yes at 260, then the process continues to make another determination at 264 as to if a new, alternate API function is available for use. If no at 262, then the process continues to 266 where a warning is generated. The warning can be generated by the AMS 102 and sent to one or more application servers and/or client devices, as well as provided to the machine learning system 122 as input data for one or more machine learning models operating on machine learning system 122.

If yes at 262, then a new, alternate API is selected for use by the application. In some embodiments, the new API may be selected by changing a lookup table, swapping a shared library, selecting the new API by a middleware function within the application, modifying a URL, or any other suitable technique now known, or hereinafter developed. The process may then continue to 266 where a warning is generated. In this instance, the warning may also indicate that a new API is being used by the application.

Below is an example pseudocode for a middleware API wrapper in accordance with embodiments of the present invention:

Middleware-DoSometing()
{
H = Get_APIHealth(default_function)
M = Get_APIHealth(alternate_function)
if ((H<HEALTH_THRESHOLD) AND (M > HEALTH_THRESHOLD))
{
altnernate_function()
}
else
{
default_function()
}
}

In the aforementioned example, a default function is used if its health metric is above the predetermined HEALTH_THRESHOLD. If the default function has a health metric below the predetermined HEALTH_THRESHOLD, and the alternate function has a health metric above the predetermined HEALTH_THRESHOLD, then the alternate function is used. In this example, if both the default function and the alternate function have a health API metric below the predetermined HEALTH_THRESHOLD, then the default function is used. The behavior can be customized using the middleware function, and other behaviors are possible in embodiments of the present invention. Thus, embodiments can include selecting a new application programming interface function in response to determining that the API metric is below the predetermined threshold. In some embodiments, the deselected API is put in to a test mode, where it is periodically invoked by a test application, and its API health is continued to be monitored. If the API health is restored to level above the predetermined threshold, then the original API may be reinstated automatically in some embodiments.

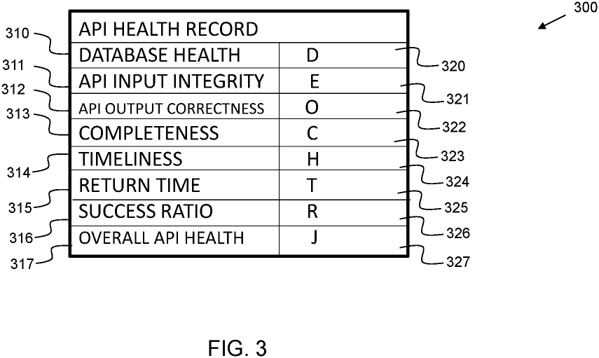

FIG. 3 shows an exemplary API health record 300 in accordance with embodiments of the present invention. The API health record can include a variety of factors, as well as an overall API health metric. In embodiments, the API health record 300 can include a database health field 310, having a value D at 320. In embodiments, the value D can range from 0 to 100, where a higher value indicates a higher (better) database health. Values other than 0 and 100 can be used as minimum and maximum values in some embodiments.

In embodiments, the API health record 300 can include an API input integrity field 311, having a value E at 321. In embodiments, the value E can range from 0 to 100, where a higher value indicates a higher (better) API input integrity. Values other than 0 and 100 can be used as minimum and maximum values in some embodiments. [0001] In embodiments, the API health record 300 can include an API output correctness field 312, having a value O at 322. In embodiments, the value O can range from 0 to 100, where a higher value indicates a higher (better) API output data correctness. Values other than 0 and 100 can be used as minimum and maximum values in some embodiments.

In embodiments, the API health record 300 can include a completeness field 313, having a value C at 323. In embodiments, the value C can range from 0 to 100, where a higher value indicates a higher (better) API output data completeness. Values other than 0 and 100 can be used as minimum and maximum values in some embodiments.

In embodiments, the API health record 300 can include a timeliness field 314, having a value H at 324. In embodiments, the value H can range from 0 to 100, where a higher value indicates a higher (better) API output data timeliness. Values other than 0 and 100 can be used as minimum and maximum values in some embodiments.

In embodiments, the API health record 300 can include a return time field 315, having a value T at 325. In embodiments, the value T can be a number of milliseconds, where a lower value indicates a shorter (better) API return time. Other time units besides milliseconds can be used in some embodiments.

In embodiments, the API health record 300 can include a success ratio 316, having a value R at 326. In embodiments, the ratio may be multiplied by a constant having a value of 100 to have the value of R ranging from 0 to 100. Thus, in embodiments, the value R can range from 0 to 100, where a higher value indicates a higher (better) API success ratio. Values other than 0 and 100 can be used as minimum and maximum values in some embodiments.

The API health record 300 can also include an overall API health 317, having a value J at 327, where J is an API health metric indicative of overall API health.

Embodiments can be used in a wide variety of applications. These applications can include cloud-based applications, distributed computing applications, and standalone computing applications. As an example, a distributed application may use an API to obtain weather information for an area. Input data for the API can include a latitude and longitude pair. Output data can include a current weather forecast for the area, as well as a future forecast for the next five days. Using this example, the various API health factors can be described. If the weather data returned is for the wrong geographical area (i.e., weather that does not correspond to the area described by the input latitude and longitude), then that is an example of degraded API output correctness 312.

If the weather data returned is missing the second and third days of the forecast, that is an example of degraded API completeness 313. If the weather data returned is from the previous week, instead of current and/or future weather, then that is an example of degraded API timeliness 314. If the API takes considerably longer to return than expected, then that can be an example of a degraded return time 315. If the API returns a non-OK error code, that can be an example of a degraded success ratio 316. If the API does not return an error when an invalid latitude or longitude is used as input, then that is an example of degraded API input integrity 311. In another example, instead of a latitude longitude pair, a location code, such as a ZIP code may be used to specify a location for the weather. A database, such as 114 of FIG. 1 may have tables or columns that relate a latitude and longitude to a ZIP code. If it is determined that there are ZIP codes that are missing from the database and/or invalid ZIP codes in the database, then that is an example of degraded database health 310. This example serves to illustrate an application for embodiments of the present invention. Other embodiments may use more, fewer, and/or different factors as compared to those shown in FIG. 3.

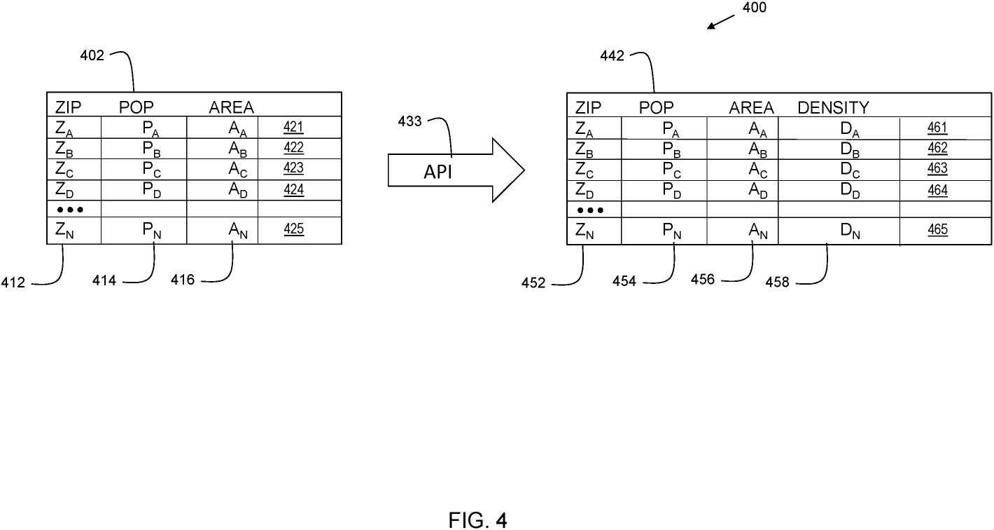

FIG. 4 shows additional data structures 400 in accordance with embodiments of the present invention. Reference to the data structures 400 is made by columns and rows. A first data structure 402 includes input data for an API. Column 412 contains ZIP code data. Column 414 contains population data. Column 416 contains area data. In table 402, rows 421, 422, 423, 424, and 425 are shown. In practice there can be more or fewer rows than shown in FIG. 4. In some cases, there can be many thousands of rows. A row and a column can be used to define a data entry. As an example, column 412, row 421 denotes a data field Z_{A}, which is a zip code. Similarly, column 414, row 423 denotes a data field Pc, which is a population. Similarly, column 416, row 425 denotes a data field A_{N}, which is an area. In this example, an API 433 uses the data in data structure 402, performs processing, and generates a new data structure 442. Data structure 442 has column 452 which is similar to column 412 of data structure 402. Data structure 442 has column 454 which is similar to column 414 of data structure 402. Data structure 442 has column 456 which is similar to column 416 of data structure 402. Additionally, data structure 442 has a column 458 for a population density that is computed by the API 433 based on the population and area values in data structure 402. The data structure 442 has a plurality of rows, indicated as 461, 462, 463, 464, and 465. As previously indicated, a row and a column can be used to define a data entry. As an example, column 458, row 464 denotes a data field D_{D}, which is a population density for the area A_{D}, based on the population P_{D}. In some embodiments, during an API profiling step (e.g., 252 of FIG. 2), data in the output column (column 458 in this example) may be crosschecked against known values as part of a verification process. The number of errors found in the output column may be used in the computation of the API health metric. The more errors that are found, the more it degrades the API health metric. In some embodiments, the API may add new data, delete existing data, and/or modify existing data to one or more tables.

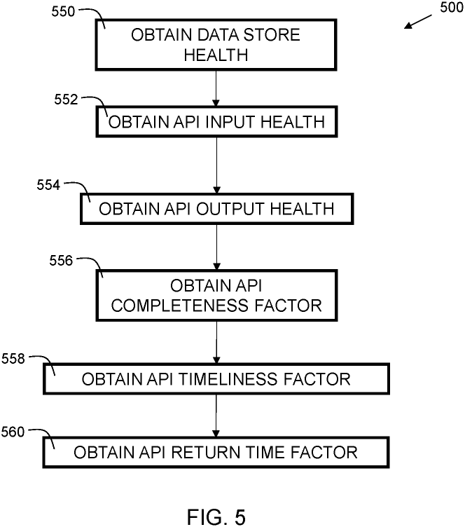

FIG. 5 is a flowchart 500 showing additional process steps for embodiments of the present invention. At 550, data store health is obtained. In embodiments, this can include determining the health of a database such as database 114 of FIG. 1. The health can include metrics indicative of levels of incorrect, missing, and/or outdated information in the database 114. At 552, an API input health is obtained. The API input health can include metrics indicative of the strictness of error checking and/or input validation for an API. In embodiments, this can be determined by using a test program to invoke an API with a variety of input data, including input data known to be erroneous, and determining if the API under test returns an appropriate error code in such situations. At 554, API output health is obtained. The API output health can include metrics indicative of the amount of incorrect data returned from the API. In embodiments, the API output health may be ascertained by inputting input values into the API that correspond to a known output value. If the output value(s) returned by the API do not match the known output value, the output data is deemed to be incorrect. Thus, some embodiments can include determining an output health factor.

At 556, an API completeness factor (metric) is obtained. The API completeness factor can include metrics indicative of how much output data is missing. As an example, if an API is supposed to return weather data for the next five days, but only returns weather data for the next three days, then that is an example of missing data. The more data that is missing, the lower the completeness factor. In some embodiments, the completeness factor C may be computed as:

$$\text{C=} \left(\text{K-M}\right) \text{/K}$$

Where K is the total amount of expected data, and M is the amount of missing data. In the aforementioned example where three days of weather data are returned when five days are expected, the completeness factor C may be computed as:

$$\text{C=} \left(\text{5-3}\right) \text{/5=2/5=0} \text{.4}$$

Thus, embodiments can include determining a completeness factor. Other formulas are possible for computing the completeness factor in some embodiments.

At 558, an API timeliness factor is obtained. The API timeliness factor can include metrics indicative of if the data is current or not. The computation of the timeliness factor may be computed based on the particular application. Some applications may include a binary computation, whereas others may include a non-binary computation. A non-binary application is one where, while it is desirable to have as current information as possible, information that is somewhat older may still be of some use. As an example, an application of a non-binary timeliness factor may include an API that returns a price for a share of stock trading on a stock exchange. The API may be designed to return stock price information that is less than 15 minutes old. If, in some invocations, the API returns stock price information that is 45 minutes old, it may still be of some use for an application. In some embodiments, the timeliness factor H may be computed as:

$$\text{H=1/max} \left(\text{1,} \left(\text{A-D+1}\right)\right)$$

Where A is the age of the returned information, and D is the target age. In the aforementioned example, the target age D is 15 minutes, and the age A of the returned information is 45. In this example, the timeliness factor H may be computed as:

$$\text{H=1/max} \left(\text{1,} \left(\text{45-15+1}\right)\right) \text{=0} \text{.3225}$$

If the age of the returned information is 17 minutes, then the timeliness factor H may be computed as:

$$\text{H} = 1 / max \left(1 , \left(\text{17-15} + 1\right)\right) = 0.333$$

If the age of the returned information is 14 minutes, then the timeliness factor H evaluates to 1, since the denominator function returns 1 based on the max function in that case.

An example application for a binary timeliness factor may include an API that returns a time when the next bus is scheduled to depart. If that returned time is in the past, then the information has no value, hence in this example, the timeliness factor H may be computed as a binary statement as indicated in the conditional notation below:

$$\text{H} = \left(R > P\right) ? 1 : 0$$

Where P is the present time, and R is the reported time, in GPS seconds. If R > P, then the reported time is in the future, and the timeliness factor is set to 1. If R is not greater than P, then the reported time is not in the future, and the timeliness factor is set to zero. Thus, some embodiments can include determining a timeliness factor. The aforementioned examples are for illustrative purposes and other timeliness factor computations and formulas are possible in embodiments of the present invention.

At 560, an API return time factor is obtained. The API return time factor can include metrics indicative of the duration required for the API to return. Some APIs, such as RESTful APIs, may generally be non-blocking calls. Other APIs may block until data is returned. The return time factor is a measure of how much time it takes for an API to return. In some embodiments, the return time factor T may be computed as:

$$\text{T} = 1 / max \left(1 , \left(\text{G} \text{-} \text{V} + 1\right)\right)$$

Where G is the actual return time in milliseconds, and V is the target return time in milliseconds. In an example where an API is intended to return within 500 milliseconds, but the actual return time is 900 milliseconds, then the return time factor T may be computed as:

$$\text{T} = 1 / max \left(1 , \left(900 \text{-} 500 + 1\right)\right) = 0.00249$$

In an example where an API is intended to return within 500 milliseconds, but the actual return time is 550 milliseconds, then the return time factor T may be computed as:

$$\text{T} = 1 / max \left(1 , \left(550 \text{-} 500 + 1\right)\right) = 0.0196$$

In an example where an API is intended to return within 500 milliseconds, but the actual return time is 350 milliseconds, then the return time factor T may be computed as:

$$\text{T = 1/ max} \left(1 , \left(350 - 500 = + 1\right)\right) = 1$$

Thus, some embodiments can include determining a return time factor. The aforementioned examples are for illustrative purposes and other return time factor computations and formulas are possible in embodiments of the present invention. In embodiments, one or more of the steps shown in FIG. 5 may be performed in a different order. In some embodiments, one or more of the steps shown in FIG. 5 may be performed concurrently.

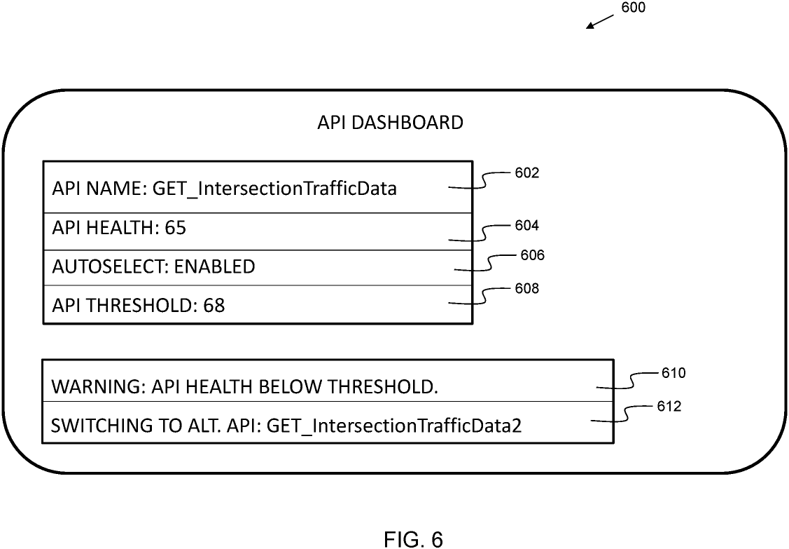

FIG. 6 is an API dashboard user interface 600 in accordance with embodiments of the present invention. The API dashboard user interface 600 conveys API health information. In embodiments, the user interface 600 may be rendered on a client computing device in communication with the AMS 102. In embodiments, the user interface 600 may be rendered as a webpage using HTML and/or other suitable protocols.

At 602, there is an API name field shown, indicating the name of an API for which health information is displayed. At 604, an API health value is shown. In embodiments, the API health may be a number ranging from 0 to 100, with a higher number indicating a better API health condition. At 606, an autoselect options is shown. The autoselect option, when enabled, allows for automatic switching to an alternate API when the health of a default API falls below a predetermined threshold. At 608, an API threshold is shown. When the autoselect is enabled, the API threshold is below which, an alternate API is substituted if available, and the API health of the alternate API is above its predetermined threshold. At 610 there is a warning message, indicating that the API health is below the threshold. At 612 there is an information message indicating switching to an alternate API.

In the example of FIG. 6, the dashboard is showing information for an API named GET IntersectionTrafficData. This may be a default API that is used by an application to get intersection traffic data, such as number of vehicles passing an intersection per hour. The API threshold is set to 68 as shown at 608, and the current API health is 65 as shown at 604. Since autoselect is enabled as shown at 606, the AMS 102 initiates a switch to an alternate API indicated as GET IntersectionTrafficData2. In some instances, the alternate API GET IntersectionTrafficData2 may accomplish a similar function as the default API GET IntersectionTrafficData, but using a different internal process, and/or data source. Using embodiments of the present invention, application functionality and reliability is maintained by automatically selecting an alternate API based on an API health value. In some embodiments, the new API may be selected by changing a lookup table, swapping a shared library, selecting the new API by a middleware function within the application, modifying a URL, or any other suitable technique now known, or hereinafter developed. The API dashboard shown in FIG. 6 is an example, and not meant to be limiting. Other API dashboard arrangements are possible in embodiments of the present invention.

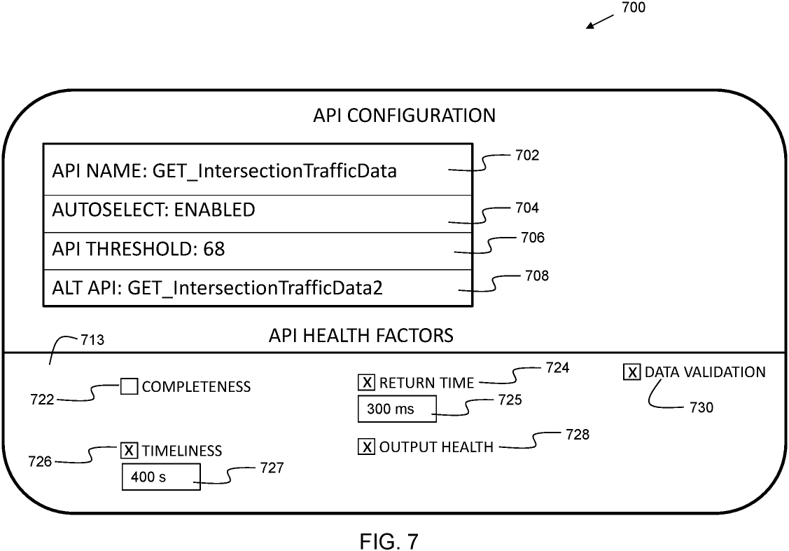

FIG. 7 is an API configuration user interface 700 in accordance with embodiments of the present invention. The API configuration user interface 700 allows a user to configure API health computations and the selection of an alternate API. In embodiments, the user interface 700 may be rendered on a client computing device in communication with the AMS 102. In embodiments, the user interface 700 may be rendered as a webpage using HTML and/or other suitable protocols.

At 702, there is an API name for the API that is to be configured. At 704, the autoselect option is configured. If the autoselect is set to enabled, then when the API health falls below a predetermined threshold, an alternate API, if available, is substituted. If autoselect is set to disabled, then API substitution does not occur. At 706, there is a field for configuring an API threshold value. Thus, in embodiments, the API configuration includes a configurable API threshold level. At 708, an alternate API is specified. When autoselect is set to enabled, and the health value of the API indicated at 702 falls below the threshold set at 706, the application will then use the alternate API specified at 708 if available. Thus, in embodiments, the API configuration includes an alternate API selection.

Section 713 allows customization of API configuration criteria used to compute the overall API health. In embodiments, the API configuration includes a plurality of selectable API health factors. In some embodiments, each API health factor of the plurality of selectable API health factors are individually selectable. In the example user interface 700, checkboxes allow selection of one or more criteria. Checkbox 722 is for completeness. Checkbox 724 is for return time. Checkbox 726 is for timeliness. Checkbox 728 is for output health, and checkbox 730 is for data validation. As shown in FIG. 7, all checkboxes are checked (marked with an “x”) except for checkbox 722. Thus, as shown in FIG. 7, completeness of output data is not used to compute the overall API health in this example. With the completeness not included, the overall API health is computed using the factors that are selected, which in the example of FIG. 7, include data validation 730, return time 724, output health 728, and timeliness 726. In some embodiments, the overall API health score is normalized such that it is within a fixed range (e.g., 0 - 100) regardless of which factors are selected and which are omitted. For timeliness 726, there is an additional field 727 to allow a user to specify a duration. In the example, a timeliness duration of 400 seconds is specified. If the API returns data that is at or less than 400 seconds old, it is considered timely. If the API returns data that is more than 400 seconds old, it is considered to be not timely, and thus degrades the overall API health.

For return time 724, there is an additional field 725 to allow a user to specify a duration. In the example, a return time duration of 300 milliseconds is specified. If the API returns within 300 milliseconds, it is considered timely. If the API takes more than 300 milliseconds to return, it is considered to be of degraded performance, and thus degrades the overall API health.

User interfaces 600 and 700 are illustrative examples, and in practice, the look can be configured in any suitable way.

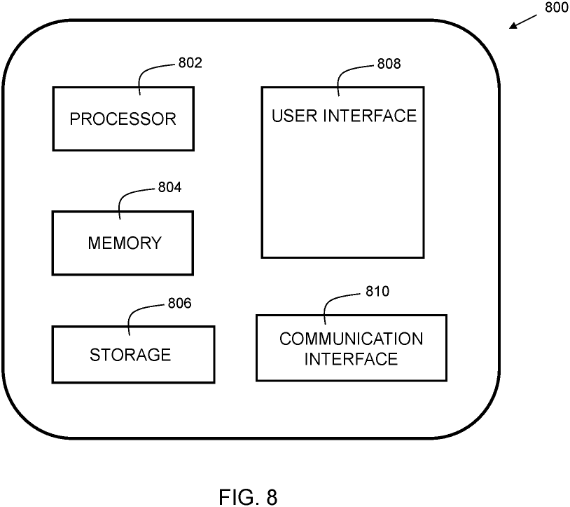

FIG. 8 shows a block diagram of an electronic device used with embodiments of the present invention that may act as a client device such as 132 of FIG. 1. Device 800 can be a smartphone, tablet computer, laptop computer, desktop computer, or other computing device. Device 800 includes a processor 802, which is coupled to a memory 804. Memory 804 may include dynamic random-access memory (DRAM), static random-access memory (SRAM), magnetic storage, and/or a read only memory such as flash, EEPROM, optical storage, or other suitable memory. In some embodiments, the memory 804 may not be a transitory signal per se.

Device 800 may further include storage 806. In embodiments, storage 806 may include one or more magnetic storage devices such as hard disk drives (HDDs). Storage 806 may additionally include one or more solid state drives (SSDs).

Device 800 further includes user interface 808. This may be a display, such as an LED display, a touch-sensitive screen, a keyboard, a mouse, or any other suitable interface for a user to interact with device 800.

The device 800 further includes a communication interface 810. The communication interface 810 may be a wired communication interface that includes Ethernet, Gigabit Ethernet, or the like. In embodiments, the communication interface 810 may include a wireless communication interface that includes modulators, demodulators, and antennas for a variety of wireless protocols including, but not limited to, Bluetooth™, Wi-Fi, and/or cellular communication protocols for communication over a computer network. The device 800 may further include other peripherals such as cameras, microphones, geolocation receives, and/or other hardware.

As can now be appreciated, disclosed embodiments improve the technical field of computer application execution and reliability. API functions are improved by evaluating their performance during a pre-deployment testing phase, as well as continuous monitoring during the deployed life of an API. Embodiments may include automatic selection of an API based on a computed API health metric. In this way, computer applications can provide improved reliability and performance using embodiments of the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method, comprising:
generating an API health record for an application programming interface API, the API health record having a factor score for each of a plurality of API health factors, the plurality of API health factors including a database health, an API input integrity, an API output correctness, a completeness, a timeliness, a return time, and a success ratio;
computing an API health metric for an API function based on factor scores from the API health record;
reporting the API health metric to a remote computing device;
determining if the API health metric is below a predetermined threshold; and
generating a message in response to determining that the API health metric is below the predetermined threshold.

2. The method of claim 1, further comprising selecting a new application programming interface function in response to determining that the API health metric is below the predetermined threshold.

3. The method of claim 1, further comprising:
performing, using a test program, a series of invocations of the API using a plurality of sets of input data, wherein the plurality of sets of input data include erroneous input data that is known to be erroneous; and
determining the API input data integrity of the API based on whether an appropriate error code is returned in response to the erroneous input data.

4. The method of claim 1, wherein the computing the API health metric includes determining a completeness factor.

5. The method of claim 1, wherein the computing the API health metric includes determining a timeliness factor.

6. The method of claim 1, wherein the computing the API health metric includes determining a return time factor.

7. The method of claim 1, wherein the plurality of sets of input data used to perform the series of invocations of the API also include known result input data having a known output value, further comprising:
determining an API output correctness based on a whether the known output value is returned in response to the known result input data.

8. The method of claim 1, further comprising obtaining an API configuration, wherein the API configuration includes a plurality of selectable API health factors.

9. The method of claim 8, wherein each API health factor of the plurality of selectable API health factors is individually selectable.

10. The method of claim 8, wherein the API configuration includes an alternate API selection.

11. The method of claim 8, wherein the API configuration includes a configurable API threshold level.

12. An electronic computation device comprising:
a processor;
a memory coupled to the processor, the memory containing instructions, that when executed by the processor, cause the electronic computation device to:
generate an API health record for an application programming interface API, the API health record having a factor score for each of a plurality of API health factors, the plurality of API health factors including a database health, an API input integrity, an API output correctness, a completeness, a timeliness, a return time, and a success ratio;
compute an API health metric for an API function based on factor scores from the API health record;
report the API health metric to a remote computing device;
determine if the API health metric is below a predetermined threshold; and generate a message in response to determining that the API health metric is below the predetermined threshold.

13. The electronic computation device of claim 12, wherein the memory further comprises instructions, that when executed by the processor, cause the electronic computation device to select a new API function in response to determining that the API health metric is below the predetermined threshold.

14. The electronic computation device of claim 12, wherein the memory further comprises instructions, that when executed by the processor, cause the electronic computation device to obtain an API configuration, wherein the API configuration includes a plurality of selectable API health factors.

15. The electronic computation device of claim 14, wherein the memory further comprises instructions, that when executed by the processor, cause the electronic computation device to make each API health factor of the plurality of selectable API health factors individually selectable.

16. The electronic computation device of claim 14, wherein the memory further comprises instructions, that when executed by the processor, cause the electronic computation device to obtain an alternate API selection.

17. The electronic computation device of claim 14, wherein the memory further comprises instructions, that when executed by the processor, cause the electronic computation device to obtain a configurable API threshold level.

18. A computer program product for an electronic computation device comprising a computer readable storage medium having program instructions embodied therewith, the program instructions, when executed by a processor , causing the electronic computation device to:
generate an API health record for an application programming interface API, the API health record having a factor score for each of a plurality of API health factors, the plurality of API health factors including a database health, an API input integrity, an API output correctness, a completeness, a timeliness, a return time, and a success ratio;
compute an API health metric for an API function based on factor scores from the API health record;
report the API health metric to a remote computing device;
determine if the API health metric is below a predetermined threshold; and
generate a message in response to determining that the API health metric is below the predetermined threshold.

19. The computer program product of claim 18, wherein the computer program product further includes program instructions, that when executed by the processor, cause the electronic computation device to select a new API function in response to determining that the API health metric is below the predetermined threshold.

20. The computer program product of claim 18, wherein the computer program product further includes program instructions, that when executed by the processor, cause the electronic computation device to obtain an API configuration, wherein the API configuration includes a plurality of selectable API health factors.
